(19) United States
(12) Patent Application Publication
(10) Pub. No.: US 2010/0330194 A1
(43) Pub. Date: Dec. 30, 2010

(54) Silicon Substituted Phosphates

(76) Inventor: Donald H. Brancato, St. Louis, MO (US)

Correspondence Address: Donald H. Brancato, 111 Calverton Road, St. Louis, MO, 63135, US

(21) Appl. No.: 12/490,579

(22) Filed: Jun. 24, 2009

(51) Int. Cl. A61K 33/42; C01B 25/12; A61P 35/00; A61P 19/08

(52) U.S. Cl. 424/601; 423/304

(57) ABSTRACT

The invention is various applications of silicon substituted phosphates of various chemical composition and granular sizes in direct application or in combination with other materials, not previously claimed in prior art used to treat the various medical conditions described.

### BACKGROUND OF THE INVENTION

1. Field of the Invention

The present invention is various medical applications of the inorganic chemicals classed as silicon substituted phosphates.

2. Description of Related Art

Many formulations of silicon substituted phosphates have been patented for use in bone related and dental applications. Patents are held by Larry Hench and related entities to whom Dr. Hench has licensed his patent. Other patents are held by medical companies such as Ortho Vita. Various researchers have presented papers at meetings, such as the Orthopaedic Research Society, where they have worked with silicon substituted phosphates formulated according to their own design.

No application of silicon substituted phosphates, prior to this invention, has been used in the applications applied for with this patent application. The biological activity of the various formulations of silicon substituted phosphates, combined with ease of use, biocompatibility, and the already approved FDA status of this material for applications other than those applied for in this application make the material especially well suited for these new medical applications.

Prior art has not used the biologic activity of silicon substituted phosphates in any of the applications of this patent application. The applications addressed in this patent application utilize several biologic characteristics of silicon substituted phosphates related to their demonstrated ability to affect cellular growth, differentiation, and secretion. Additionally, the demonstrated antimicrobial capacity of silicon substituted phosphates has not been specifically addressed by prior art.

### DESCRIPTION OF THE INVENTION

### Objects of the Invention

Accordingly, it is the object of this invention to provide a biologically active inorganic compound for use on living tissue with applications for the treatment of various medical conditions, including, but not limited to those described below, to with:
1. The invention is used to fix medical devices, such as, but not exclusive to prosthetic implants, by promoting direct soft tissue or bone adherence or ingrowth to the prosthetic surface without intermediate tissue, such as granulation tissue, being formed.
2. The invention is used to achieve regeneration of tissue of the type to which it is contact. That is to say that when in contact with muscle, muscle regenerates; when in contact with skin, skin regenerates; when in contact with vascular structures, vessels regenerate; etc.
3. Because of the material's demonstrated regenerative potentiation, combining that capacity with its demonstrated antimicrobial character, this material has applications in infected or potentially infected wounds and lesions. These include, but are not exclusive to:
   a.) the treatment and healing of diabetic and decubitus ulcers
   b.) the treatment and healing of traumatic injuries, such as, but not exclusive to battlefield wounds, motor vehicle accident injuries, etc.
4. The incorporation of the material into various bioabsorbable fixation devices, such as, but not exclusive to plates, screws, anchors, staples, interference screws, rods, etc., of various compositions, such as, but not exclusive to polylactic acid, polyglycolic acid, poly hydroalkonates, poliglecaprone, collagen preparations etc., to allow such devices to remodel into the host tissue type without the formation of granulation tissue and loosening.
5. The application of the material to bone—soft tissue interfaces to promote the formation of Sharpey's fibers providing anatomic soft tissue to bone, load transferring bonds.
6. The application of the material to fractures with a high incidence of non or delayed union to prevent non or delayed union. The material may be used in conjunction with other compatible vehicles or carriers for ease of application or injection, such as, but not exclusive to various biocompatible fluids or gels of various composition, such as, but not exclusive to polysaccharides or proteins, etc.
7. The application of the material to bleeding, non-vascular surfaces, such as bleeding bone, for control of bleeding.
8. The application of the material to treat benign bone cysts and tumors following the removal of abnormal tissue or fluid to prevent recurrence.
9. The silicon substituted phosphates are wholly and completely biocompatible.
10. The chemical and composite nature of the invention may vary according to the method of manufacture and application requirements, including, but not exclusive to granular size and proportions of chemicals composing it, all comprising a material described as silicon substituted phosphates.
11. The invention is easily handled and used in a sterile environment, such as an operating room, or otherwise any temperate environment, requiring only application to involved areas, with or without the use of special apparatus.
12. It is stable throughout a wide range of temperature.

Additional objects will, in part be obvious, and will, in part, appear hereinafter.

The invention accordingly comprises applications of silicon substituted phosphates possessing the features and properties described herein, all as exemplified in the following summary and detailed description, with the scope of the invention to be indicated in the claims.

### BRIEF SUMMARY OF THE INVENTION

The foregoing objects are attained using already commercially available methods of manufacture, providing silicon substituted phosphates that collectively provide a sterile, surgically implantable material for the regeneration of living tissues of various composition, to allow tissue fixation to dissimilar tissue and other materials, to treat various medical conditions, and to promote clotting. The invention, originally a sterile, dry composite, may be applied directly to open or accessible sites, mixed with other vehicles or carriers, or incorporated into other devices. This material chemically modulates various cellular growth and differentiation factors such as, but not exclusive to tumor necrosis factor, to promote tissue regeneration of the anatomic type that it is placed in contact with, while exhibiting antimicrobial activity.

### DETAILED DESCRIPTION OF THE PREFERRED EMBODIMENTS

This invention is various applications of silicon substituted phosphates.

In one embodiment, silicon substituted phosphates, in various granular sizes and chemical compositions dependent on the processes of manufacture, may sprinkled or otherwise directly applied to involved areas or other components of treating modalities, such as but not exclusive to wound sites, prosthetics, tissue interfaces, debrided benign bone cysts and tumors, etc.

A second embodiment sees the material in combination with a vehicle or carrier to facilitate the presentment of the invention to various sites, in some instances to less accessible areas by injection, etc

At third embodiment sees the invention combined in various proportions with other materials to facilitate remodeling of those various bioabsorbable devices.

A fourth embodiment sees the invention applied to bleeding bone to promote clotting, and stop bleeding from non-vascular tissues. It may be added to or combined with various carriers, such as, but not exclusive to, precipitated collagen mixed as a paste with other clotting agents such as, but not exclusive to, aqueous thrombin; or other bioabsorbable sealants; and applied to bleeding bone surfaces for the control of bleeding.

From the above, it is clearly evinced that the invention represents a versatile, unique, and reliable advancement to multiple areas of medical and surgical treatment for a variety of conditions and circumstances.

The terms and expressions herein used are used as terms of description and not of limitation; and there is no intention in the use of such terms and expressions of excluding any equivalents or alternatives to the composition, features, portions, and descriptions of the invention or its composition. Therefore, it is recognized that various modifications are possible within the scope of the invention claimed.

What is claimed is:

1. Applications of silicon substituted phosphates: the invention comprising:
A. Silicon substituted phosphates of various chemical composition and granular sizes directly applied or mixed with other materials to various sites to:
a. promote prosthetic fixation
b. prevent prosthetic loosening from particulates and other causes
c. promote tissue regeneration of tissue of the type the material is applied to
d. treat benign bone cysts and tumors after debridement to prevent recurrence
e. promote clotting on non-vascular tissues
B. Silicon substituted phosphates of various chemical composition and granular sizes applied to treat diabetic and decubitus ulcers
C. Silicon substituted phosphates of various chemical composition and granular sizes applied to surgically formed tissue interfaces to regenerate load transferring bonds between different tissue types
D. Silicon substituted phosphates of various chemical composition and granular sizes used with vehicles or carriers to facilitate the application of the material
E. Silicon substituted phosphates of various chemical composition and granular sizes combined with other materials to form bioabsorbable devices able to remodel into the host tissue type without loosening, granulation formation, etc.
